(19) United States
(12) Patent Application Publication
(10) Pub. No.: US 2008/0190435 A1
(43) Pub. Date: Aug. 14, 2008

(54) Infant nasal cannula

(76) Inventor: Conrad Hansen, Pasadena, CA (US)

Correspondence Address: JAMES E. BRUNTON, ESQ., P. O. BOX 29000, GLENDALE, CA, 91209, US

(21) Appl. No.: 11/707,456

(22) Filed: Feb. 14, 2007

(51) Int. Cl. A61M 15/08

(52) U.S. Cl. 128/207.18

(57) ABSTRACT

A nasal cannula apparatus that is particularly well suited for use with infants and small children. The apparatus includes a soft, close fitting knitted cap that supports elongated flexible oxygen tubes in a comfortable and secure manner about the infant's head. The knitted cap readily adjusts to the size of the infant's head and effectively remains in position about the infant's head as the infant moves about in its crib or infant bed.

### BACKGROUND OF THE INVENTION

1. Field of the Invention

The present invention relates generally to breathing devices. More particularly, the invention relates to a uniquely configured nasal cannula device for use by infants and small children.

2. Description of the Prior Art

Nasal cannulas are well known in the art. Typically these prior art cannulas comprise a pair of elongated flexible nasal tubes each connected at a one end to a nasal end portion and connected at the other end to a source of pressurized oxygen.

Typically, once the nasal end portions have been inserted into the patient's nose, the flexible nasal tubes are draped over the patient's ears and then extend down the patient's back to a point of interconnection with an oxygen supply. Over time, the nasal tubes severely irritate the patient's ears and cause substantial discomfort.

This problem has been uniquely solved by the present inventor by providing a nasal cannula apparatus that totally eliminates the need for draping the nasal tubes over the ears. More particularly, in the apparatus devised by the present inventor, the flexible nasal tubes are interconnected with a soft expandable headband which encircles a user's head and can be comfortably worn by the patient as the oxygen is being administered. One form of this novel nasal cannula apparatus is illustrated and described in U.S. Design Pat. No. D479,327 S.

While the apparatus illustrated and described in Design Pat. No. D479,327 is well suited for use by adults and older children it has proved not to be particularly well suited for use by infants and small children because of the difficulty in maintaining the headband in position about the infant's head as he infant moves about in its crib. It is this difficulty that is uniquely overcome by the apparatus of the present invention, the character of which will be described in the paragraphs which follow.

### SUMMARY OF THE INVENTION

It is an object of the present invention to provide a nasal cannula apparatus that is of a unique design that is particularly well suited for use with infants and small children. More particularly, in this novel apparatus a soft, close fitting knitted cap takes the place of the headband of applicant's earlier invention and functions to provide a superior means for supporting the elongated flexible oxygen tubes in a comfortable and secure manner about the infant's head.

Another object of the invention is to provide an apparatus of the aforementioned character in which the knitted cap readily adjusts to the size of the infant's head and effectively remains in position about the infant's head as the infant moves about in its crib or infant bed.

Another object of the invention is to provide a nasal cannula apparatus of the character described in which strategically positioned connectors are affixed to either side of the knitted cap and proximate the crown of the cap for use in securing the oxygen tubes to the cap in a manner to avoid discomfort to the infant.

Another object of the invention is to provide an apparatus as described in the preceding paragraphs which is easy to manufacture, is easy-to-use in both the home and hospital environments and one that is relatively trouble-free in use.

The foregoing as well as other objects of the invention will be realized by the apparatus of the invention the details of which will be more fully described in the paragraphs that follow.

### BRIEF DESCRIPTION OF THE DRAWINGS

FIG. 1 is a generally perspective view of one form of the infant nasal cannula apparatus of the present invention shown in position over the head of an infant.

FIG. 2 is a fragmentary, side-elevational view of the apparatus shown in FIG. 1.

FIG. 3 is an enlarged, cross-sectional view taken along lines 3-3 of FIG. 1.

FIG. 4 is an enlarged, cross-sectional view taken along lines 4-4 of FIG. 2.

FIG. 5 is a cross-sectional view taken along lines 5-5 of FIG. 3.

FIG. 6 is an enlarged, cross-sectional view taken along lines 6-6 of FIG. 3.

### DESCRIPTION OF THE INVENTION

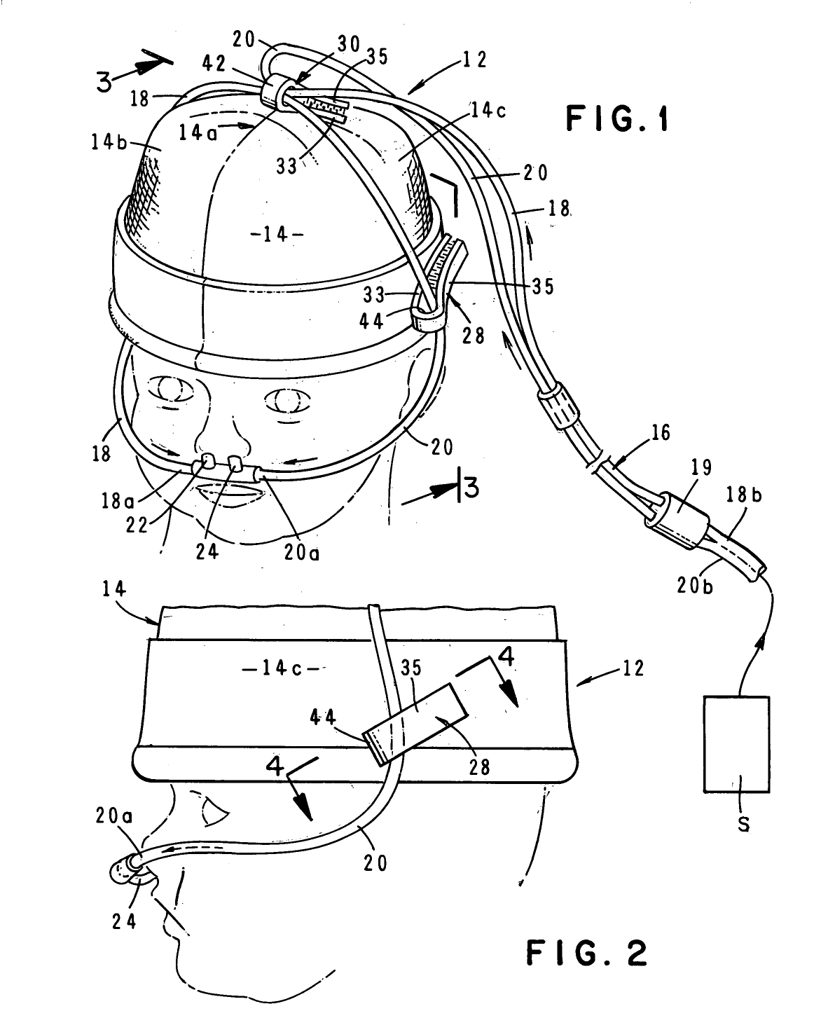

Referring to the drawings and particularly to FIGS. 1 and 2, one form of the infant cannula apparatus of the present invention is there illustrated and generally designated by the numeral 12. This apparatus, which is specially designed for use with infants and small children, comprises a close fitting cap 14 and nasal cannula 16 that is connected to the cap in the manner illustrated in FIG. 1. Nasal cannula 16 comprises first and second elongated flexible nasal tubes 18 and 20. As best seen in FIG. 1, end 18a of tube 18 is connected to a nasal end portion 22 and is connected at its other end 18b to a connector 19, which is, in turn, connected to a source of pressurized oxygen Similarly, end 20a of tube 20 is connected to a nasal end portion 24 and is connected at its other end 20b to connector 19.

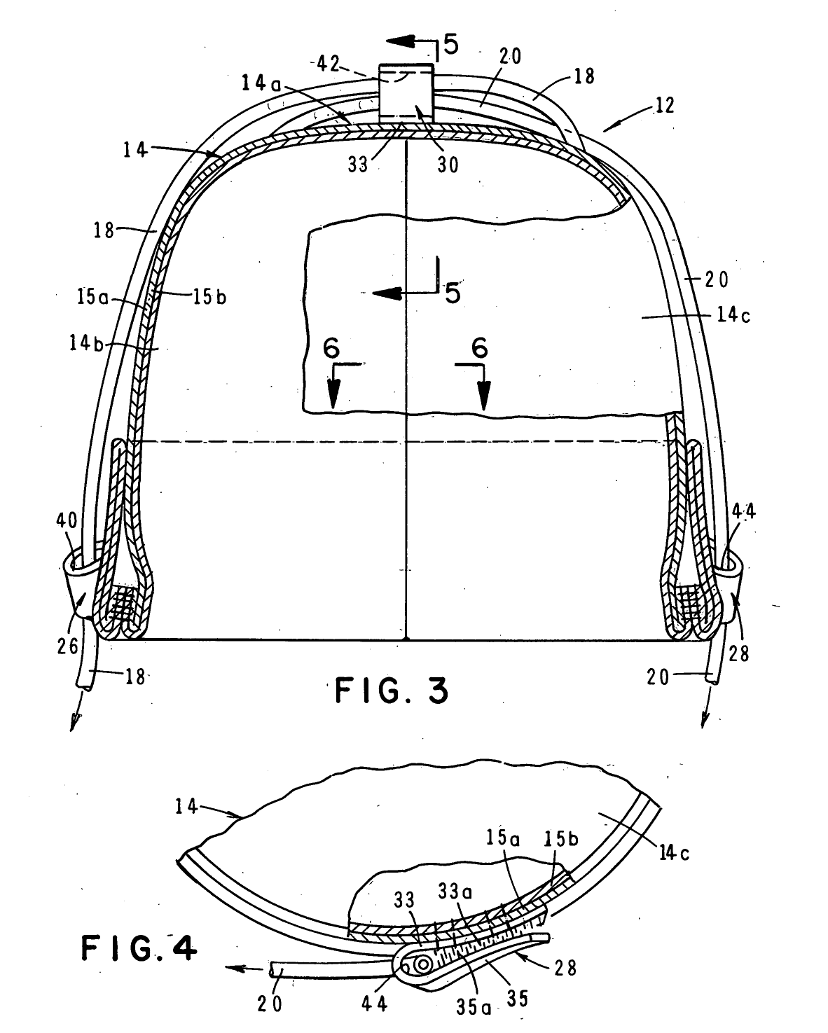

As indicated in FIG. 1, cap 14, which is preferably constructed from an expandable, contractible knitted or woven material, is specially adapted to be closely fitted over the infant's head. Cap 14, which is here made up of inner and outer interconnected layers 15a and 15b (FIGS. 3 and 6), here includes an upper crown portion 14a and first and second side portions 14b and 14c respectively.

Connected to cap 14 are a plurality of strategically located connectors for removably interconnecting the elongated flexible nasal tubes 18 and 20 to the cap. More particularly, these important connectors here comprises a first Velcro strip 26 that is connected to the first side 14b of the cap, a second Velcro strip 28 that is connected to said second side 14c of the cap and a third Velcro strip 30 that is connected to the crown 14a of the cap (FIG. 4).

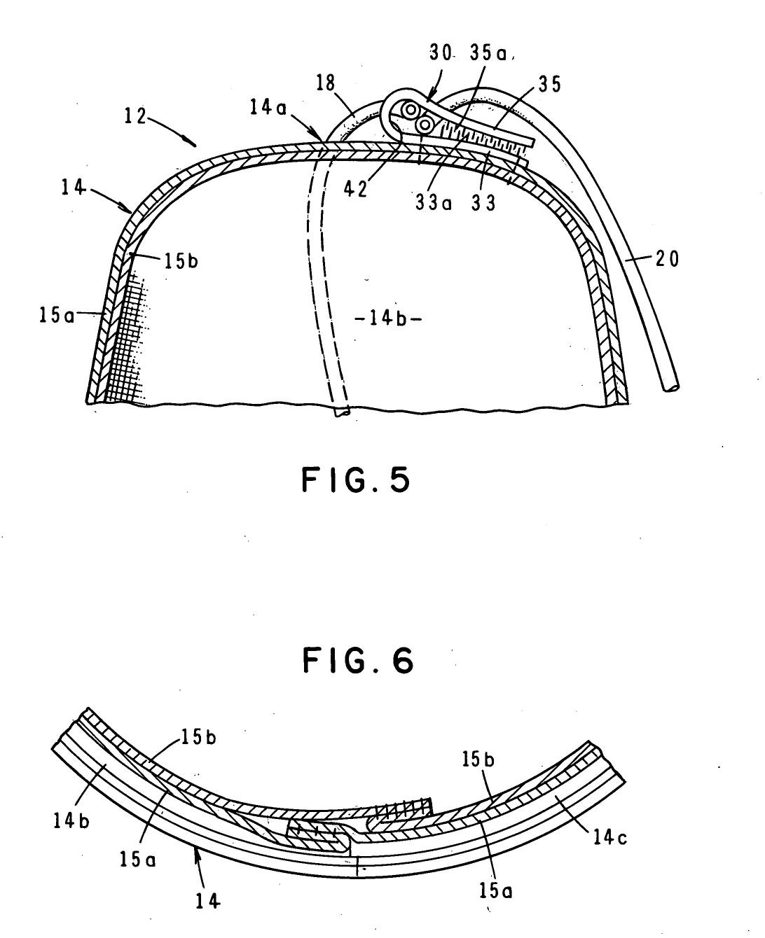

Each of the strips 26, 28 and 30 has a first portion 33 that is connected to the cap as by sewing and a second portion 35 that is provided with a Velcro material 35a. As best seen in FIGS. 4 and 5, Velcro material 35a can be releasably secured to a Velcro material 33a that is provided on first portion 33 to form a loop portion adapted to receive the elongated flexible nasal tubes. With this construction, after the nasal end portions 22 and 24 have been inserted into the infants nasal passageways, tube 18 can be passed around one side of the infants face in the manner shown in FIG. 1, through loop 40 (see FIG. 3) and then upwardly along side 14b of the cap. At the crown 14a of the cap, tube 18 can be secured within loop 42 and then extended downwardly for interconnection with the oxygen source “S”. In similar fashion, tube 20 can be passed around the other side of the infant's face in the manner shown in FIG. 1, secured within loop 44 and then passed upwardly along the opposite side 14c of the cap. At the crown 14a of the cap, tube 20 can be also secured within loop 42 in a crossing relationship with tube 18 and then extended downwardly for interconnection with the oxygen source “S”.

When the novel apparatus of the invention is in use, the soft, close fitting knitted cap functions to provide a superior means for supporting the elongated flexible oxygen tubes in a comfortable and secure manner about the infant's head. Because the knitted cap readily adjusts to the size of the infant's head, it effectively remains in position about the infant's head as the infant moves about in its crib or infant bed.

Having now described the invention in detail in accordance with the requirements of the patent statutes, those skilled in this art will have no difficulty in making changes and modifications in the individual parts or their relative assembly in order to meet specific requirements or conditions. Such changes and modifications may be made without departing from the scope and spirit of the invention, as set forth in the following claims.

I claim:

1. A nasal cannula apparatus for use with infants and small children, including a nasal cannula having a pair of elongated flexible nasal tubes each connected at one end to a nasal end portion and connected at the other end to a source of pressurized oxygen, said apparatus further comprising:
(a) a cap constructed to be closely fitted over the infant's head, said cap having a crown portion and first and second side portions; and
(b) a plurality of connectors connected to said cap for removably interconnecting the pair of elongated flexible nasal tubes to said cap.

2. The apparatus as defined in claim 1 in which said plurality of connectors comprises a first Velcro strip connected to said first side of said cap, a second Velcro strip connected to said second side of said cap and a third Velcro strip connected to said crown of said cap.

3. The apparatus as defined in claim 1 in which said cap is constructed from an expandable, contractible knitted material.

4. The apparatus as defined in claim 1 in which said cap is constructed from an expandable, contractible woven material.

5. A nasal cannula apparatus for use with infants and small children, including a nasal cannula having a pair of elongated flexible nasal tubes each connected at a one end to a nasal end portion and connected at the other end to a source of pressurized oxygen, said apparatus further comprising:
(a) an expandable, contractible cap constructed to be closely fitted over the infant's head, said cap having a crown portion and first and second side portions; and
(b) a plurality of connectors connected to said cap for removably interconnecting the pair of elongated flexible nasal tubes to said cap, said plurality of connectors comprising a first Velcro strip connected to said first side of said cap, a second Velcro strip connected to said second side of said cap and a third Velcro strip connected to said crown of said cap.

6. The apparatus as defined in claim 5 in which said cap is constructed from an expandable, contractible knitted material.

7. The apparatus as defined in claim 5 in which said cap is constructed from an expandable, contractible woven material.
